(19) United States
(12) Patent Application Publication
(10) Pub. No.: US 2014/0270067 A1
(43) Pub. Date: Sep. 18, 2014

(54) RADIOGRAPHIC MARKER

(71) Applicant: Stephen Knecht Clark, Salt Lake City, UT (US)

(72) Inventor: Stephen Knecht Clark, Salt Lake City, UT (US)

(21) Appl. No.: 13/830,134

(22) Filed: Mar. 14, 2013

(51) Int. Cl. A61B 19/00

(52) U.S. Cl. CPC A61B 19/54
USPC 378/62; 378/163

(57) ABSTRACT

A radiographic marker. The marker includes a first portion of radiolucent material comprising a first radiopaque pattern. The first portion is configured to be positioned on a first surface of an imaging subject during use. The marker also includes a second portion of radiolucent material comprising a second radiopaque pattern visually distinguishable from the first radiopaque pattern. The second portion is configured to be positioned on a second generally opposite surface of the subject during use. When radiant energy is emitted through the first portion, the subject, and the second portion to produce a radiograph, the first radiopaque pattern and the second radiopaque pattern produce corresponding first and second shadows on the radiograph. The first shadow and the second shadow are visually distinguishable from one another based on the first radiopaque pattern being visually distinguishable from the second radiopaque pattern, and are usable for identifying characteristics of the radiograph.

### CROSS-REFERENCE TO RELATED APPLICATIONS

Not Applicable.

### BACKGROUND

1. Field of the Invention

The present application relates to radiographic markers for use in radiographic imaging.

2. Background and Relevant Art

Radiographic imaging involves generation of radiographic images (radiographs) of a subject (e.g., body part) by capturing the shadows cast by the subject on a radiographic detector (e.g., film, digital sensor, etc.) during emission of radiant energy (e.g., x-ray, gamma ray, etc.) through the subject. For example, two-dimensional radiographic imaging has been revolutionary in the field of medicine, by providing two-dimensional representations of three-dimensional anatomy, such as bone, tooth, etc. These two-dimensional radiographs can be used for diagnosis, treatment, etc. For example, in the field of dentistry, two-dimensional radiographs of tooth and jaw structures are used to assist and guide a practitioner when performing procedures such as drilling, implants, etc. by providing the practitioner a snapshot of the subject(s) on which the practitioner is operating.

Despite all of its benefits, two-dimensional radiographic imaging does have some shortcomings. For example, the process of generating two-dimensional radiographs of three-dimensional subjects often introduces distortions in the resulting image. Distortions may result from the angle of the radiant energy source relative to the radiographic detector, bending of the radiographic detector (e.g., in the case of a film detector), distance of the radiant energy source from the radiographic detector, thickness of the subject, misalignment of the radiographic detector and the subject, etc. Some mechanisms have been developed for interpreting some distortions, such as magnification. For example, to interpret magnification a radiopaque object (e.g., a metal ball) of a known size is included in the radiograph, and the size of the shadow cast by the object is used to identify the absolute magnitude of magnification. However, such objects do not communicate any additional information, such as a magnification that also includes an elongation, the relative location of the radiant energy source, etc.

In light of the distorted nature of two-dimensional radiographic imaging, practitioners have increased accuracy of radiography by capturing multiple radiographs and comparing the radiographs to one another. However, doing so can be costly in terms of lab technician time and supply costs (e.g., film costs). In addition, capturing multiple radiographs increases the exposure of the person being imaged to harmful radiant energy.

Practitioners have also increased accuracy of radiography by capturing three-dimensional radiographs, such as through the use of computerized tomography (CT) scanning. However, CT scanning equipment and trained personnel are very expensive, and the CT scanning process exposes the person being imaged to very high levels of harmful radiant energy.

### BRIEF SUMMARY

At least some embodiments described herein relate to radiographic markers for use in radiographic imaging, which communicate information about conditions existing at the time of capture of the radiograph (e.g., alignment of imaging equipment) as well as information about depth within a two-dimensional radiograph, and methods, kits, and products related thereto. The embodiments described herein enable a practitioner more accurately interpret the subject of a radiograph and to capture more accurate radiographs.

In some embodiments, a radiographic marker includes a first portion of radiolucent material comprising a first radiopaque pattern. The first portion is configured to be positioned on a first surface of an imaging subject during use. The radiographic marker also includes a second portion of radiolucent material that comprises a second radiopaque pattern that is visually distinguishable from the first radiopaque pattern. The second portion is configured to be positioned on a second generally opposite surface of the subject during use. When a radiograph is produced by emitting radiant energy through the first portion, the subject, and the second portion, the first radiopaque pattern and the second radiopaque pattern produce corresponding first and second shadows on the radiograph. The first shadow and the second shadow are visually distinguishable from one another based on the first radiopaque pattern being visually distinguishable from the second radiopaque pattern. The shadows are usable for identifying characteristics of a subject as shown on the radiograph.

In some embodiments, a method for capturing a radiograph includes positioning a first portion of radiolucent material on a first surface of an imaging subject, and positioning a second portion of radiolucent material on a second generally opposite surface of the imaging subject. The first portion of radiolucent material includes a first radiopaque pattern, and the second portion of radiolucent material includes a second radiopaque pattern that is visually distinguishable from the first radiopaque pattern. The method also includes capturing a radiograph by emitting radiant energy through the first portion of radiolucent material and the second portion of radiolucent material, causing the first radiopaque pattern to cast a first shadow on a radiographic detector and causing the second radiopaque pattern to cast a second shadow on the radiographic detector. The method also includes interpreting one or more image characteristics of the radiograph based on identification of the first shadow as corresponding to the first radiopaque pattern, based on identification of the second shadow as corresponding to the second radiopaque pattern, and based on a relative alignment of the first shadow and the second shadow.

In some embodiments, a radiograph includes a first shadow having a first shape. The first shadow was placed on the radiograph in response to positioning a first portion of radiolucent material, which includes a first radiopaque pattern that generates the first shadow when radiant energy is passed there through, on a first surface of an imaging subject. The radiograph also includes a second shadow having a second shape. The second shadow was placed on the radiograph in response to positioning a second portion of radiolucent material, which includes a second radiopaque pattern that is visually distinguishable from the first radiopaque pattern and that generates the second shadow when radiant energy is passed there through, on a second opposite surface of the imaging subject. The first shadow and the second shadow are usable for interpreting one or more characteristics of the subject as shown on the radiograph, based on identification of the first shadow as corresponding to the first radiopaque pattern, based on identification of the second shadow as corresponding to the second radiopaque pattern, and based on a relative alignment of the first shadow and the second shadow.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1A illustrates a flexible radiographic marker, according to one or more embodiments of the present invention.

FIG. 1B illustrates a rigid radiographic marker, according to one or more embodiments of the present invention.

FIG. 1C illustrates a multi-part radiographic marker comprising different physically separated portions, according to one or more embodiments of the present invention.

FIG. 2A illustrates the flexible radiographic marker of FIG. 1A in a flexed configuration, according to one or more embodiments of the present invention.

FIG. 2B illustrates the flexible radiographic marker of FIG. 1A in a flexed configuration, according to one or more embodiments of the present invention.

FIG. 3 illustrates capture of a radiograph using a radiographic marker, according to one or more embodiments of the present invention.

FIG. 4 illustrates a radiograph that includes a radiographic marker, according to one or more embodiments of the present invention.

FIG. 5 illustrates a flowchart of a method for capturing a radiograph, according to one or more embodiments of the present invention.

FIG. 6 illustrates example variations of radiographic marker patterns to create a perception of depth, according to one or more embodiments of the present invention.

FIG. 7 illustrates example variations of radiographic marker patterns, according to one or more embodiments of the present invention

FIG. 8A illustrates a variation of the rigid radiographic marker of FIG. 1B, according to one or more embodiments of the present invention.

FIG. 8B illustrates a variation of the rigid radiographic marker of FIG. 1B, according to one or more embodiments of the present invention.

FIG. 8C illustrates a variation of the rigid radiographic marker of FIG. 1B, according to one or more embodiments of the present invention.

FIG. 8D illustrates a variation of the rigid radiographic marker of FIG. 1B, according to one or more embodiments of the present invention.

FIG. 9 illustrates a kit of radiographic markers, according to one or more embodiments of the present invention.

FIG. 10 illustrates an alignment guide for use with radiographic markers, according to one or more embodiments of the present invention.

### DETAILED DESCRIPTION

At least some embodiments described herein relate to radiographic markers for use in radiographic imaging, which communicate information about conditions existing at the time of capture of the radiograph (e.g., alignment of imaging equipment) as well as information about depth within a two-dimensional radiograph, and methods, kits, and products related thereto. The embodiments described herein enable a practitioner more accurately interpret the subject of a radiograph and to capture more accurate radiographs.

The radiographic markers described herein are comprised of a radiolucent material, and enable a practitioner to apply a marker portion or segment (e.g., a first and second portion) to at least two opposite sides of an imaging subject. Each marker portion includes one or more corresponding radiopaque patterns, which are configured to cause attenuation of radiant energy, and to cast differing shadows on a radiographic detector during radiographic imaging by virtue of the attenuation. The differing shadows are usable by a practitioner to ascertain conditions that existed at the time of radiographic imaging, such as the angle of a radiant energy source relative to the subject and/or the radiographic detector, characteristics of the subject as shown on the radiograph, such as magnification (both absolute magnification and elongation of the magnification), etc.

In some embodiments, at least two marker portions each include a different element of a crosshair and target pattern (e.g., a circle that forms an outer target mark and an “X” or a cross that forms an inner crosshair for alignment with the target mark). Alignment of the crosshair and target pattern indicate accuracy of the alignment of the radiographic detector, the imaging subject, and the radiant energy source, and can be used for interpretation of the subject of the radiograph, and/or for making corrections when capturing subsequent radiographs. In some embodiments, at least two marker portions each include a pattern (e.g., dots or lines/dashes) that provide the perception of depth and also indicate alignment of the radiographic detector, the imaging subject, and the radiant energy source.

In some embodiments, radiographic markers include an additional (e.g., third) marker portion that is applied to a side of the imaging subject that is generally perpendicular to the opposite sides of the subject to which the first and second portions are applied. The additional marker portion can include one or more additional radiopaque patterns, such as a pattern indicating a center point and/or centerline of the radiographic marker.

Radiographic markers according to embodiments of the present invention can comprise flexible markers configured to be flexed about three or more sides of an imaging subject. Radiographic markers according to embodiments of the present invention can also comprise rigid markers that are configured to be positioned around three or more sides of an imaging subject. Radiographic markers according to embodiments of the present invention can also comprise physically separated portions of a multi-part marker that are configured to be adhered to differing sides of an imaging subject.

Radiographic markers may be comprised of any appropriate flexible or rigid radiolucent material, such as paper, fabric (e.g., silk, polyester), film, rubber, acrylic, silicone, wood, polymers (e.g., polymers used in dental impressions), etc. In some embodiments, radiographic markers are comprised of a single radiolucent material, while in other embodiments radiographic markers are comprised of a combination of different radiolucent materials. As used herein, a radiolucent material includes any material that allows radiant energy (e.g., x-ray, gamma ray) to pass there though without substantial attenuation.

Radiopaque patterns may be comprised of any appropriate radiopaque material that substantially blocks or attenuates radiant energy, sufficient to cast a visually detectable shadow on a radiographic detector. Examples of radiopaque materials include metal and radiopaque inks Metal radiopaque materials may include washers, wires, thread, metal tracks (e.g., etched), etc. Radiopaque patterns may be applied to a surface of a radiographic marker (e.g., as in the case of radiopaque inks), or may be embedded within the radiolucent material of a radiographic marker.

While the embodiments of radiographic markers and use thereof is now described herein in the context of dentistry, one of skill in the relevant art will recognize the radiographic markers described herein, or variants thereof, may be used in practically any field of radiography. As such, the description herein and the appended claims are not limited to radiographic markers and use thereof in the field of dentistry.

FIG. 1A illustrates a flexible radiographic marker 100, according to one or more embodiments of the present invention. Flexible radiographic marker 100 can comprise any appropriate flexible radiolucent material, such as paper, fabric (e.g., silk, polyester), film, rubber, etc. Flexible radiographic marker 100 includes a plurality of portions or segments, including a first portion 101a, a second portion 101b, and a third portion 101c. The exact dimensions and boundaries of each portion/segment may vary, but each portion is generally structured to be positioned about a different side of an imaging subject. In dental use, for example, when the subject (e.g., a tooth or jawbone) is in the anterior portion of a person's mouth, portion 101a may be positioned about the lingual side of a tooth and/or jawbone, portion 101b may be positioned about the facial side of the tooth and/or jawbone, and portion 101c may be positioned about the occlusal side of the tooth and/or jawbone.

As depicted, flexible radiographic marker 100 includes radiopaque patterns forming unique and contrasting pattern images. The particular patterns depicted are for illustrative purposes only. One of skill in the art will recognize that different patterns may be used, and that radiographic markers may include only a subset of the depicted radiopaque patterns, or may include one or more additional radiopaque patterns. Additional embodiments of radiopaque patterns embodied on flexible radiographic marker 100 are depicted in FIGS. 6 and 7. Such patterns may be embodied on any form of radiographic marker (e.g., flexible, rigid, multi-part).

The depicted radiopaque patterns include a target mark pattern 102a, and a corresponding crosshair pattern 102e. When flexible radiographic marker 100 is flexed about its center point in perfect alignment with a radiant energy source and a radiographic detector, target mark pattern 102a and crosshair pattern 102e form a crosshair pattern as a shadow on a radiograph (see FIG. 2A). If there is a misalignment between target mark pattern 102a and crosshair pattern 102e, or between the radiant energy source and the radiographic detector, target mark pattern 102a and crosshair pattern 102e may form a misaligned shadow on a radiograph (see FIG. 2B). The relative alignment of shadows caused by target mark pattern 102a and crosshair pattern 102e can be used to identify distortions in the radiograph, such as the nature of the misalignment.

The depicted radiopaque patterns also include a dot pattern 102b, and a corresponding line pattern 102d. Dot pattern 102b and line pattern 102d are configured to cast shadows that provide the perception of depth in the radiograph (see FIGS. 2A and 2B). As such, dot pattern 102b and line pattern 102d enable interpretation of what is close and what is far in the subject, and a perception of a depth of field. While dot pattern 102b and line pattern 102d are depicted as being fully embodied on portions 101a and 101b, they may also extend onto portion 101c.

The depicted radiopaque patterns also include a center pattern 102c. Center pattern 102c is configured to provide a reference for the center of flexible radiographic marker 100. When flexible radiographic marker 100 is positioned about a center point and centerline of the imaging subject, center pattern 102c also casts a shadow that can be used to identify the center point and centerline of the imaging subject.

Flexible radiographic marker 100 may be secured to the imaging subject in a variety of manners. For example, if flexible radiographic marker 100 is comprised of fabric or paper, flexible radiographic marker 100 may be secured to an imaging subject though the clinging properties these materials have when wet. As such, flexible radiographic marker 100 may be secured with water, saliva, saline, etc. Additionally or as an alternative, flexible radiographic marker 100 may include an adhesive, or have an adhesive applied thereto, that can be used to adhere flexible radiographic marker 100 to the imaging subject. In some embodiments, flexible radiographic marker 100 may be in the form of an adhesive tape.

Flexible radiographic marker 100 may be embodied in a variety of dimensions, including different thicknesses (i.e., thickness of the radiolucent material), different widths (i.e., along dimension 100a), and different lengths (i.e., along dimension 100b). As such, flexible radiographic marker 100 may be dimensioned for different purposes, such as for marking different teeth in the mouth, for marking teeth only (i.e., excluding the jawbone), for marking the jawbone (i.e., excluding teeth), for marking both teeth and the jawbone, etc. For example, for dental use, the typical length (along dimension 100b) of flexible radiographic marker 100 may vary from about 5 mm for some smaller imaging subjects to about 50 mm for some larger imaging subjects. The typical width (along dimension 100a) of flexible radiographic marker 100 may vary from about 3 mm to about 15 mm.

FIG. 1B illustrates a rigid (or semi-rigid) radiographic marker 103, according to one or more embodiments of the present invention. Rigid radiographic marker 103 can comprise any appropriate rigid (or semi-rigid) radiolucent material, such as plastic, acrylic, silicone, rubber, etc. Rigid radiographic marker 103 includes a plurality of portions or segments, including a first portion 104a, a second portion 104b, and a third portion 104c. The exact dimensions of each portion may vary, but each portion is generally structured to be positioned about a different side of an imaging subject. In dental use, for example, when the subject (e.g., a tooth or jawbone) is in the anterior portion of a person's mouth, portion 104a may be positioned about the lingual side of a tooth and/or jawbone, portion 104b may be positioned about the facial side of the tooth and/or jawbone, and portion 104c may be positioned about the occlusal side of the tooth and/or jawbone.

As depicted, rigid radiographic marker 103 includes radiopaque patterns forming unique and contrasting pattern images. The particular patterns depicted are for illustrative purposes only. One of skill in the art will recognize that different patterns may be used, and that radiographic markers may include only a subset of the depicted radiopaque patterns, or may include one or more additional radiopaque patterns. In general, the patterns used on flexible radiographic marker 100 may also be applied to rigid radiographic marker 103.

The depicted radiopaque patterns include a target mark pattern 105a, and a corresponding crosshair pattern 105e. When rigid radiographic marker 103 in perfect alignment with a radiant energy source and a radiographic detector, target mark pattern 105a and crosshair pattern 105e form a crosshair within the target mark shadow on a radiograph. If there is a misalignment, target mark pattern 105a and crosshair pattern 105e may be misaligned on a radiograph.

The depicted radiopaque patterns also include a dot pattern 105b, and a corresponding line pattern 105d. Dot pattern 105b and line pattern 105d are configured to provide the perception of depth in the radiograph. As such, dot pattern 105b and line pattern 105d enable interpretation of what is close and what is far in the subject, and a perception of a depth of field. In the depicted embodiment, dot pattern 105b and line pattern 105d extend onto center portion 104c.

The depicted radiopaque patterns also include a center pattern 105c. Center pattern 105c is configured to provide a reference for the center of rigid radiographic marker 103. When rigid radiographic marker 103 is positioned about a center point and centerline of the imaging subject, center pattern 105c also casts a shadow that can be used to identify the center point and centerline of the imaging subject.

Rigid radiographic marker 103 may be secured to the imaging subject through used of adhesives, gravity, biting, etc. In dental use, for example, radiographic marker 103 may be positioned over a subject tooth or gum portion, and may be secured by the patient biting down on the marker.

The rigid radiographic marker 103 may be embodied in a variety of dimensions, including different thickness of radiolucent material, as well as different lengths (i.e., along dimension 103b) and widths (i.e., along dimension 103c) of portions 104a and 104b and different lengths (i.e., along dimension 103a) and widths (i.e., along dimension 103c) of center portion 104c. As such, rigid radiographic marker 103 may be dimensioned for different purposes, such as for marking different teeth in the mouth, for marking teeth only (excluding the jawbone), for marking the jawbone (excluding teeth), for marking the teeth and the jawbone, etc. Examples of different dimensions/configurations of rigid radiographic marker 103 are provided in FIGS. 8A-8D.

FIG. 1C illustrates a multi-part radiographic marker 106 that includes a plurality of physically separated marker portions or segments, according to one or more embodiments of the present invention. For example, multi-part radiographic marker 106 may comprise a plurality of different “stickers” that are adhered to different sides of an imaging subject.

Multi-part radiographic marker 106 can comprise any appropriate radiolucent material, such as paper, fabric, film, rubber, etc. Multi-part radiographic marker 106 includes at least two physically separated portions, including a first portion 107a and a second portion 107b, which are configured to be secured to opposite sides the imaging subject. The radiographic marker may also include one or more additional portions, such as a third portion 107c, which is configured to be secured to an additional side of the imaging subject. The exact dimensions of each portion may vary, but each portion is generally structured to be positioned about a different side of the imaging subject. In dental use, for example, when the subject (i.e., a tooth or jawbone) is in the anterior portion of a person's mouth, portion 107a may be positioned about the lingual side of a tooth and/or jawbone, portion 107b may be positioned about the facial side of the tooth and/or jawbone, and portion 107c may be positioned about the occlusal side of the tooth and/or jawbone.

As depicted, multi-part radiographic marker 106 includes radiopaque patterns forming unique and contrasting pattern images. The particular patterns depicted are for illustrative purposes only. One of skill in the art will recognize that different patterns may be used, and that radiographic markers may include only a subset of the depicted radiopaque patterns, or may include one or more additional radiopaque patterns. In general, the patterns used on flexible radiographic marker 100 may also be applied to multi-part radiographic marker 106.

The depicted radiopaque patterns include a target mark pattern 108a, and a corresponding crosshair pattern 108e. When multi-part radiographic marker 106 is in perfect alignment with a radiant energy source and a radiographic detector, the target mark pattern 108a and the crosshair pattern 108e form a crosshair within the target mark shadow on a radiograph. If there is a misalignment, the target mark pattern 108a and the crosshair pattern 108e may be misaligned on a radiograph. The depicted radiopaque patterns also include a dot pattern 108b, and a corresponding line pattern 108d. The dot pattern 108b and the line pattern 108d are configured to provide the perception of depth in the radiograph. As such, dot pattern 108b and line pattern 108d enable interpretation of what is close and what is far in the subject, and a perception of a depth of field. The depicted radiopaque patterns also include a center pattern 108c. The center pattern is configured to provide a reference for a center point and centerline of the imaging subject. While dot pattern 108b and line pattern 108d are depicted as being fully embodied on portions 107a and 107b, one or both of the patterns may extend onto center portion 107c.

Multi-part radiographic marker 106 may be secured to the imaging subject in a variety of manners through clinging properties, adhesives, etc. Each portion of multi-part radiographic marker 106 may be embodied in a variety of dimensions, including different thickness of radiolucent material, and different widths (i.e., along dimension 106a) and different lengths (i.e., along dimension 106b) of each separated portion.

Accordingly, embodiments include an apparatus comprising a radiographic marker. The radiographic marker includes a first portion of radiolucent material that includes a first radiopaque pattern and that is configured to be positioned on a first surface of an imaging subject during use. For example, flexible radiographic marker 100 can include portion 101b with one or both of pattern 102d and pattern 102e. In another example, rigid radiographic marker 103 can include portion 104b with one or both of pattern 105d and pattern 105e. In yet another example, multi-part radiographic marker 106 can include portion 107b with one or both of pattern 108d and pattern 108e. Portion 101b, portion 104b, or portion 107b can be positioned on a first side of an imaging subject (e.g., the facial side of a tooth).

The radiographic marker also includes a second portion of radiolucent material that includes a second radiopaque pattern and that is configured to be positioned on a second generally opposite surface of the imaging subject during use, wherein the first radiopaque pattern is visually distinguishable from the second radiopaque pattern. For example, flexible radiographic marker 100 can include portion 101a with one or both of pattern 102a and pattern 102b. In another example, rigid radiographic marker 103 can include portion 104a with one or both of pattern 105a and pattern 105b. In yet another example, multi-part radiographic marker 106 can include portion 107a with one or both of pattern 108a and pattern 108b. Portion 101a, portion 104a, or portion 107a can be positioned on a second opposite of an imaging subject (e.g., the lingual side of a tooth).

When a radiograph is produced by emitting radiant energy through the first portion of radiolucent material, the subject, and the second portion of radiolucent material, the first radiopaque pattern and the second radiopaque pattern produce corresponding first and second shadows on a radiograph that are usable for identifying one or more characteristics of the subject as shown on the radiograph. The first shadow and the second shadow are visually distinguishable from one another based on the first radiopaque pattern being visually distinguishable from the second radiopaque pattern. For example, radiant energy may pass through portion 101b, the imaging subject, and portion 101a (in either direction); through portion 104b, the imaging subject, and portion 104a (in either direction); or through portion 107b, the imaging subject, and portion 107a (in either direction). The patterns on these portions will cause a plurality of shadows to be formed on the radiograph, which are usable for identifying one or more characteristics of the radiograph.

The radiographic marker apparatus can also include one or more additional portions, such as portion 101c of flexible radiographic marker 100, portion 104c of rigid radiographic marker 103, or portion 107c of multi-part radiographic marker 106. These respective portions can also cast shadows on an imaging subject, providing additional information about the conditions that existed at the time of capture of the radiograph, such as the center point of the marker, or the center point and/or centerline of the imaging subject.

FIG. 3 illustrates a configuration, within the context of dentistry, for capture of a radiograph using a radiographic marker. As depicted, a radiant energy source 300 emits radiant energy 301 (e.g., x-ray, gamma ray) at a radiographic detector 302 (e.g., film, digital sensor). Within the path of radiant energy 301 is an imaging subject 303 (e.g., a tooth). A radiographic marker 304 (e.g., flexible radiographic marker 100) has been positioned over imaging subject 303, including having a portion of radiographic marker 304 being positioned over opposite sides of imaging subject 303 within the path of radiant energy 301 (e.g., portion 101a over the lingual side of the tooth and portion 101b over the facial side of the tooth), and having a portion of radiographic marker 304 being positioned over a side of imaging subject 303 that is generally perpendicular to the opposite sides (e.g., portion 101c over the occlusal side of the tooth). As such, as radiant energy 301 passes through the imaging subject 303 and each portion of radiographic marker 304, the radiopaque patterns on radiographic marker produce shadows on radiographic detector 302 which are perceptible on the radiograph.

FIG. 4 illustrates a radiograph 400, such as one that may be captured using the configuration of FIG. 3. FIG. 4 illustrates a two-dimensional representation of shadows cast by the imaging subject 303, as well as shadows cast by radiographic marker 304. For example, FIG. 4 illustrates a shadow 401 cast by target mark pattern 102a, a shadow 402 cast by crosshair pattern 102e, a shadow 403 cast by line pattern 102d, a shadow 404 cast by dot pattern 102b, and a shadow 405 cast by center pattern 102c.

The alignment of shadow 401 (target mark) and shadow 402 (crosshair) helps a viewer interpret the angle of radiant energy source 300 relative to radiographic detector 302, and also provides a perception of depth in the radiograph. The sizes of shadow 401 and shadow 402 can also be used to ascertain magnification of the subject. For example, by corresponding shadow 401 and shadow 402 with the respective sizes of target mark pattern 102a and crosshair pattern 102e on radiographic marker 304. Knowledge of the differences in size between target mark pattern 102a and crosshair pattern 102e, and perception of the size of their shadows (401, 402) can enable a viewer to interpret depth, and to interpret magnification, including both magnitude and elongation (e.g., three-dimensions). The alignment of shadows 403 and 404 also provide a perception of depth in the image, and can also be used to interpret the angle of radiant energy source 300 relative to radiographic detector 302.

Accordingly, embodiments of the present invention can include a radiograph that includes a first shadow having a first shape. The first shadow is placed on the radiograph in response to positioning a first portion of radiolucent material on a first surface of an imaging subject. The first portion of radiolucent material includes a first radiopaque pattern that generates the first shadow when radiant energy is passed through the first portion of radiolucent material. The radiograph also includes second shadow having a second shape that is visually distinguishable from the first shape. The second shadow is placed on the radiograph in response to positioning a second portion of radiolucent material on a second opposite surface of the imaging subject. The second portion of radiolucent material includes a second radiopaque pattern that is visually distinguishable from the first radiopaque pattern and that generates the second shadow when radiant energy is passed through the second portion of radiolucent material. The first shadow and the second shadow are usable for interpreting one or more characteristics of the subject of the radiograph based on identification of the first shadow as corresponding to the first radiopaque pattern, based on identification of the second shadow as corresponding to the second radiopaque pattern, and based on a relative alignment of the first shadow and the second shadow.

FIG. 5 illustrates a method 500 for capturing a radiograph. Method 500 comprises an act of positioning a radiographic marker on an imaging subject (act 501). Act 501 can include positioning a first portion of radiolucent material on a first surface of an imaging subject, the first portion of radiolucent material including a first radiopaque pattern. For example, portion 101b of the flexible radiographic marker 100 may be positioned on a first surface of a tooth (e.g., the facial side). In another example, portion 104b of the rigid radiographic marker 103 may be positioned on the first surface of the tooth. In yet another example, portion 107b of multi-part radiographic marker 106 may be positioned on the first surface of the tooth. The portion positioned on the first surface of the tooth can contain one or more radiopaque patterns, such as patterns 102d and/or 102e (portion 101b), patterns 105d and/or 105e (portion 104b), or patterns 108d and/or 108e (portion 107b).

Act 501 can also include positioning a second portion of radiolucent material on a second generally opposite surface of the imaging subject, the second portion of radiolucent material including a second radiopaque pattern that is visually distinguishable from the first radiopaque pattern. For example, portion 101a of the flexible radiographic marker 100 may be positioned on a second surface of a tooth (e.g., the lingual side). In another example, portion 104a of the rigid radiographic marker 103 may be positioned on the second surface of the tooth. In yet another example, portion 107a of multi-part radiographic marker 106 may be positioned on the second surface of the tooth. The portion positioned on the second surface of the tooth can contain one or more radiopaque patterns, such as patterns 102a and/or 102b (portion 101a), patterns 105a and/or 105b (portion 104a), or patterns 108a and/or 108b (portion 107a).

The patterns on the portion of the marker positioned on the second surface of the tooth are visually distinguishable from the patterns on the portion of the marker positioned on the first surface of the tooth, and cast shadows (e.g., x-ray, gamma ray shadows) corresponding to those patterns. With respect to flexible radiographic marker 100, for example, pattern 102a is visually distinguishable from pattern 102e, and pattern 102b is visually distinguishable from pattern 102d. With respect to rigid radiographic marker 103, pattern 105a is visually distinguishable from pattern 105e, and pattern 105b is visually distinguishable from pattern 105d. With respect to multi-part radiographic marker 106, pattern 108a is visually distinguishable from pattern 108e, and pattern 108b is visually distinguishable from pattern 108d.

Method 500 may also include positioning a third portion of a radiographic marker on a third surface of the imaging subject, such as a surface that is generally perpendicular to the first and second surfaces. For example, portion 101c of flexible radiographic marker 101 may be placed on the occlusal side of the tooth, portion 104c of rigid radiographic marker 103 may be placed on the occlusal side of the tooth, or portion 107c of multi-part radiographic marker 106 may be placed on the occlusal side of the tooth.

Method 500 also comprises an act of capturing a radiograph (act 502). Act 502 can include capturing a radiograph by emitting radiant energy through the first portion of radiolucent material and the second portion of radiolucent material, causing the first radiopaque pattern to cast a first shadow on a radiographic detector and causing the second radiopaque pattern to cast a second shadow on the radiographic detector, the first shadow and the second shadow being usable for interpreting one or more image characteristics of the radiograph based on identification of the first shadow as corresponding to the first radiopaque pattern, based on identification of the second shadow as corresponding to the second radiopaque pattern, and based on a relative alignment of the first shadow and the second shadow. For example, a radiant energy source (e.g., radiant energy source 300) can emit radiant energy that proceeds through the first portion of the radiographic marker, the imaging subject, and the second portion of the radiographic marker (in either direction), casting shadows on the radiographic detector. Radiant energy may also pass through a third portion of the radiographic marker, casting an additional shadow on the radiographic detector, such as a shadow indicating a center point and/or centerline of the radiographic marker and/or the imaging subject.

In the example of flexible radiographic marker 100, radiant energy may pass through portion 101b, the imaging subject, and portion 101a, although the reverse ordering may also be used. Radiant energy may also pass through portion 101c. The resulting radiograph may be similar to the radiograph of FIG. 4, with shadow 401 being visually distinguishable from shadow 402, shadow 403 being visually distinguishable from shadow 404, and shadow 405 marking the top and/or center of the imaging subject. Similar energy paths and resulting radiographs may be achieved through use of rigid radiographic marker 103 and multi-part radiographic marker 106.

The first and second shadow can be used to ascertain several characteristics of the radiograph, and the conditions that existed at the time of capture of the radiograph. For example, the first shadow and second shadow can correspond to an outer portion of a target pattern (e.g., a circle) and an inner portion of the target pattern (e.g., a cross), and can be used to ascertain the position of the radiant energy source based on the relative alignment of the outer portion of the target pattern and the inner portion of the target pattern.

In another example, the first shadow and the second shadow can be used for ascertaining a magnification distortion of the radiograph based on the size of the first shadow and/or the size of the second radio shadow. For example, the first shadow and second shadow can correspond to an outer portion of a target pattern (e.g., a circle) and an inner portion of the target pattern (e.g., a cross). The outer and inner portions can have a known size on the radiographic marker, and the size of the shadows cast by these patterns can be used to ascertain magnification distortion of the radiograph in three dimensions (e.g., by comparing the size of the first and second shadow, with knowledge of the size of the outer and inner portions on the marker).

In another example, the first shadow and the second shadow are usable for providing a perception of depth of field in the radiograph, and for interpretation near and far subject elements, based on the relative alignment of the first shadow and the second shadow. For example, alignment of a dot pattern (e.g., pattern 102b) and a line pattern (e.g., pattern 102d) can provide the perception of depth.

Capture and interpretation of radiographs may be assisted by or automated through use of computers. For example, a computer may be configured to identify the shadows in a radiograph, and to correspond these shadows with known patterns on the radiographic marker used when capturing the radiograph. By identifying the differences between shadows in the radiograph, and through knowledge of the physical characteristics of the patterns generating these shadows, the computer system can identify one or more distortions in the radiograph. In addition, by identifying the differences between shadows in the radiograph, and through knowledge of the physical characteristics of the patterns generating these shadows, the computer system may also identify one or more instructions for capturing a less-distorted radiograph. These identified distortions and/or instructions can be communicated to a user. For example, FIG. 10 illustrates some exemplary mappings between target alignment and how to move the radiant energy source (e.g., x-ray tube or head) to increase target alignment. In addition, the computer system may digitally alter (e.g., rotate, skew, etc.) the radiograph to compensate for the distortions, provide instructions for obtaining a more accurate radiograph, or automatically re-align image capture equipment to compensate for the distortions when capturing subsequent radiographs.

Use of computers in connection with the radiographic markers described herein can include accessing a radiograph that includes a plurality of shadows that are visually distinguishable from one another and that were produced using a radiographic marker that includes contrasting or visually distinguishable radiopaque patterns. Accessing a radiograph can include accessing a previously captured radiograph (e.g., previously captured by a human or by a computer system), or providing electronic instructions for capturing a radiograph using computer-controlled equipment.

Use of computers in connection with the radiographic markers described herein can also include identifying a plurality of visually distinguishable shadows in the radiograph. For example, the computer system may identify shadows corresponding to patterns 102a and 102e and/or shadows corresponding to patterns 102b and 102d. The computer system may also identify shadows marking the middle/centerline of a marker and/or an imaging subject (e.g., pattern 102c).

Use of computers in connection with the radiographic markers described herein can also include correlating the identified shadows with known patterns of a radiographic marker. For example, a computer system may identify that a first identified shadow corresponds to marker 102a, and that a second identified shadow corresponds to marker 102e.

Use of computers in connection with the radiographic markers described herein can also include, based on the identified shadows and the correlation between the shadow and patterns of a radiographic marker, identifying one or more distortions in the radiograph, and/or identifying one or more conditions that existed at the time of capture of the radiograph. For example, the computer system may identify magnification (absolute and elongated), the relationships between the imaging target, a radiant energy source, and a radiographic detector, etc.

Use of computers in connection with the radiographic markers described herein can also include one or more of informing a user about a distortion, performing image manipulation for correcting the distortion, proving a user instructions for capturing a new radiograph in a manner that addresses the distortion, sending instructions for reconfiguring radiographic capture equipment, sending electronic instructions for capturing a new radiograph, etc.

FIGS. 6 and 7 illustrate some radiopaque pattern variations. While these variations are depicted as being applied to flexible radiographic marker 100, these variations or derivatives thereof may be applied to any radiographic marker (e.g., rigid radiographic marker 103 or multi-part radiographic marker 106). FIG. 6 depicts a plurality of pattern variations that are each configured to create shadows that provide the perception of depth in different a manner. For example, the pattern variations include varying the width of lines in patterns (i.e., compare patterns 601, 602, and 603, in which the lines are of varying widths). By varying the width of lines in a single radiograph, the shadows cast by these lines help to emphasize the perception of depth of field.

The pattern variations also include using a crosshair portion of a crosshair pattern having an overall size that varies from an overall size of a target mark portion of the crosshair pattern (i.e., compare the size of target mark portion 605 and crosshair portion 604). For example, varying the size of the target mark portion and the crosshair portion can also enhance the perception of depth of field. Furthermore, knowledge of the respective sizes of these marks, along with measurement of the sizes of their shadows, provides a way to measurably compute depth in the subject image of the radiograph. In some embodiments, the diameter of target mark portion 605 and crosshair portion 604 may vary from about 1 mm to about 8 mm.

FIG. 7 illustrates that other variations could include varying the density of lines and/or dots, and providing a centerline through the lines and/or dots. Other variations may include using different weights (i.e., thickness) of lines and/or dots, using different shapes of elements of the crosshair pattern (e.g., squares, triangles, etc.), or any other variation that contrasts/distinguishes the shadow produced by one pattern from the shadow produced by another pattern, and which can be used to identify depth, magnification, and other characteristics of a radiograph.

FIGS. 8A-8D illustrate some example variations of rigid radiographic marker 103. FIG. 8A illustrates that rigid radiographic marker 103 can include one or more rounded corners to increase patient comfort during use of rigid radiographic marker 103. FIG. 8B illustrates that rigid radiographic marker 103 can include angled and/or flexible portions that can help position rigid radiographic marker 103 over opposite sides of an imaging subject when the opposite sides are not completely parallel. In some embodiments, each portion (104a, 104b, 104c) is constructed from a rigid material, but the portions are connected using a flexible material. FIGS. 8C and 8D illustrate that rigid radiographic marker 103 can be configured in various sizes, including variations in thickness of radiolucent material, and length and width of each portion/segment. Differing sizes of rigid radiographic marker 103 can enable a practitioner to select a marker that best fits the imagining subject, as described previously in connection with FIG. 1B.

FIG. 9 illustrates that radiographic markers can be distributed as part of a kit. Each kit can include a plurality of radiographic markers having varying dimensions. As such, a practitioner can select a radiographic marker suitable for the imaging subject being captured with a radiograph. Each kit can also include markers of varying types (e.g., flexible, rigid, multi-part), and can include markers having differing patterns. Depending on the material used in the radiographic markers, the radiographic markers may be single-use or multi-use. For example, a flexible radiographic marker made of paper or fabric may be single-use, whereas a radiographic marker (flexible or rigid) made of plastic, film, or rubber may be multi-use, either with multiple imaging subjects of the same patient or with multiple imaging subjects of different patients. If designed as a multi-use radiographic marker, the radiographic marker may be constructed from materials that are easily sanitized. A kit may include radiographic markers of both single-use and multi-use varieties, or may include only radiographic markers of a single use type (single-use or multi-use).

In some embodiments, a kit may include a plurality of physically separated radiographic markers provided in a container, such as a box (as shown in FIG. 9). In some embodiments a kit may comprise a roll of adhesive tape that includes a plurality of radiographic markers that are cut or torn apart. In some embodiments a kit may comprise a sheet that has a plurality of radiographic markers adhered thereto.

Accordingly, embodiments may include a kit for radiographic imaging. The kit may include a plurality of radiographic markers. Each radiographic marker may comprise (i) a first portion of radiolucent material that includes a first radiopaque pattern and (ii) a second portion of radiolucent material that includes a second radiopaque pattern that is visually distinguishable from the first radiopaque pattern. The first and second portion of each radiographic marker is configured to be placed on opposite sides of a subject during use, and to cast shadows on a radiographic detector. The shadows include a first shadow corresponding to the first radiopaque pattern and a second shadow corresponding to the second radiopaque pattern.

In some embodiments, the first and second portion of a first radiographic marker of the plurality of radiographic markers has dimensions differing from the first and second portion of a second radiographic marker of the plurality of radiographic markers. As such, the kit includes radiographic markers of differing sizes.

In some embodiments, a first radiographic marker of the plurality of radiographic markers is of a different type than a second radiographic marker of the plurality of radiographic markers. For example, the kit may include a combination of flexible, rigid, and/or multi-part markers. Additionally or alternatively, the kit may include markers of different material types (e.g., paper, fabric, plastic, film, rubber, silicone). Additionally or alternatively, the kit may include markers of different use types (e.g., single-use or multi-use).

FIG. 10 an alignment guide for use with radiographic markers. The alignment guide is expressed in terms of a target mark pattern (e.g., pattern 102a) and a crosshair pattern (e.g., pattern 102e). The alignment guide provides a practitioner with immediate feedback with respect to how to reposition a radiant energy source (e.g., x-ray tube or head) to correct misalignment between the radiant energy source, radiographic detector, and the radiographic marker. As such, the practitioner is able to receive timely feedback about the quality of a radiograph, and how to improve the quality of subsequent radiographs. For example, if a target mark and a crosshair are touching, it may indicate that the radiographic detector and the radiant energy source are within a specified alignment (e.g., 3 degrees), and can help direct the magnitude of movement of the radiant energy source, the radiographic detector, and/or the radiographic marker that is necessary to correct the misalignment.

Accordingly, embodiments of the present invention can greatly enhance the usability and the accuracy information that is obtained from a two-dimensional radiograph, mitigating the need to capture a large number of radiographs, or to employ more expensive and more damaging three-dimensional imaging techniques (e.g., CT scanning) In addition, embodiments of the present invention attach permanent documentation (distinguishable shadows) to a radiograph that is usable for ascertaining the conditions that existed at the time of capture of the radiograph.

Embodiments of the present invention may comprise or utilize a special-purpose or general-purpose computer system that includes computer hardware, such as, for example, one or more processors and system memory, as discussed in greater detail below. Embodiments within the scope of the present invention also include physical and other computer-readable media for carrying or storing computer-executable instructions and/or data structures. Such computer-readable media can be any available media that can be accessed by a general-purpose or special-purpose computer system. Computer-readable media that store computer-executable instructions and/or data structures are computer storage media. Computer-readable media that carry computer-executable instructions and/or data structures are transmission media. Thus, by way of example, and not limitation, embodiments of the invention can comprise at least two distinctly different kinds of computer-readable media: computer storage media and transmission media.

Computer storage media are physical storage media that store computer-executable instructions and/or data structures. Physical storage media includes recordable-type storage devices, such as RAM, ROM, EEPROM, solid state drives (“SSDs”), flash memory, phase-change memory (“PCM”), optical disk storage, magnetic disk storage or other magnetic storage devices, or any other physical storage medium which can be used to store program code in the form of computer-executable instructions or data structures, and which can be accessed by a general-purpose or special-purpose computer system.

Transmission media can include a network and/or data links which can be used to carry program code in the form of computer-executable instructions or data structures, and which can be accessed by a general-purpose or special-purpose computer system. A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer system, the computer system may view the connection as transmission media. Combinations of the above should also be included within the scope of computer-readable media.

Further, upon reaching various computer system components, program code in the form of computer-executable instructions or data structures can be transferred automatically from transmission media to computer storage media (or vice versa). For example, computer-executable instructions or data structures received over a network or data link can be buffered in RAM within a network interface module (e.g., a “NIC”), and then eventually transferred to computer system RAM and/or to less volatile computer storage media at a computer system. Thus, it should be understood that computer storage media can be included in computer system components that also (or even primarily) utilize transmission media.

Computer-executable instructions comprise, for example, instructions and data which, when executed at one or more processors, cause a general-purpose computer system, special-purpose computer system, or special-purpose processing device to perform a certain function or group of functions. Computer-executable instructions may be, for example, binaries, intermediate format instructions such as assembly language, or even source code.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The invention may also be practiced in distributed system environments where local and remote computer systems, which are linked (either by hardwired data links, wireless data links, or by a combination of hardwired and wireless data links) through a network, both perform tasks. As such, in a distributed system environment, a computer system may include a plurality of constituent computer systems. In a distributed system environment, program modules may be located in both local and remote memory storage devices.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

I claim:

1. A radiographic marker, comprising:
a first portion of radiolucent material that includes a first radiopaque pattern and that is configured to be positioned on a first surface of an imaging subject during use; and
a second portion of radiolucent material that includes a second radiopaque pattern and that is configured to be positioned on a second generally opposite surface of the imaging subject during use, wherein the first radiopaque pattern is visually distinguishable from the second radiopaque pattern,
wherein, when a radiograph is produced by emitting radiant energy through the first portion of radiolucent material, the subject, and the second portion of radiolucent material, the first radiopaque pattern and the second radiopaque pattern produce corresponding first and second shadows on a radiograph that are usable for identifying one or more characteristics of the subject as shown on the radiograph, the first shadow and the second shadow being visually distinguishable from one another based on the first radiopaque pattern being visually distinguishable from the second radiopaque pattern.

2. The radiographic marker as recited in claim 1, further comprising a third portion of radiolucent material positioned between the first and second portions of radiolucent material, the third portion of radiolucent material comprising a third radiopaque pattern that is configured to produce a shadow on the radiograph that indicates a center of the radiographic marker.

3. The radiographic marker as recited in claim 1, wherein the radiolucent material comprises a flexible radiolucent material.

4. The radiographic marker as recited in claim 1, wherein the radiolucent material comprises a rigid radiolucent material.

5. The radiographic marker as recited in claim 1, wherein the first radiopaque pattern comprises a crosshair pattern and wherein the second radiopaque pattern comprises a target mark pattern.

6. The radiographic marker as recited in claim 1, wherein the first portion of radiolucent material also includes a third radiopaque pattern and the second portion of radiolucent material also includes a fourth radiopaque pattern, and wherein the third radiopaque pattern and the fourth radiopaque pattern are configured to generate corresponding shadows that provide a perception of depth and that enable interpretation of the imaging subject relative to a radiant energy source and a radiographic detector.

7. The radiographic marker as recited in claim 1, wherein one or both of the first radiopaque pattern or the second radiopaque pattern is comprised of radiopaque ink.

8. The radiographic marker as recited in claim 1, wherein one or both of the first radiopaque pattern or the second radiopaque pattern is comprised of a metallic material.

9. The radiographic marker as recited in claim 1, wherein the radiographic marker is part of a kit that includes at least one additional radiographic marker, the additional radiographic marker having one or more dimensions that are different from one or more dimensions of the radiographic marker.

10. The radiographic marker as recited in claim 1, wherein the radiographic marker is part of a kit that includes at least one additional radiographic marker, the additional radiographic marker being comprised of one or more radiolucent materials that are different from one or more radiolucent materials of the radiographic marker.

11. A method for capturing a radiograph, comprising:
positioning a first portion of radiolucent material on a first surface of an imaging subject, the first portion of radiolucent material including a first radiopaque pattern;
positioning a second portion of radiolucent material on a second generally opposite surface of the imaging subject, the second portion of radiolucent material including a second radiopaque pattern that is visually distinguishable from the first radiopaque pattern; and
capturing a radiograph by emitting radiant energy through the first portion of radiolucent material and the second portion of radiolucent material, causing the first radiopaque pattern to cast a first shadow on a radiographic detector and causing the second radiopaque pattern to cast a second shadow on the radiographic detector, the first shadow and the second shadow being usable for interpreting one or more image characteristics of the radiograph based on identification of the first shadow as corresponding to the first radiopaque pattern, based on identification of the second shadow as corresponding to the second radiopaque pattern, and based on a relative alignment of the first shadow and the second shadow.

12. The method as recited in claim 11, wherein the first shadow and the second shadow are usable for ascertaining a position of a radiant energy source based on a relative alignment of an outer target portion of a target and crosshair pattern and an inner crosshair portion of the target and crosshair pattern.

13. The method as recited in claim 11, wherein the first shadow and the second shadow are usable for ascertaining a magnification distortion of the radiograph based on a size of the first shadow or a size of the second shadow.

14. The method as recited in claim 11, wherein the first shadow and the second shadow are usable for ascertaining depth based on the relative alignment of the first shadow and the second shadow.

15. The method as recited in claim 11, wherein the first shadow and the second shadow are usable for ascertaining depth based on a size of the first shadow relative to a size of the second shadow.

16. The method as recited in claim 11, wherein the first portion of radiolucent material also includes a third radiopaque pattern and the second portion of radiolucent material also includes a fourth radiopaque pattern, and wherein the third radiopaque pattern and the fourth radiopaque pattern are configured to generate corresponding shadows that provide a perception of depth and that enable interpretation of the imaging subject relative to a radiant energy source and a radiographic detector.

17. The method as recited in claim 11, further comprising positioning a third portion of radiolucent material on a third surface of the imaging subject, the third portion of radiolucent material comprising a third radiopaque pattern that is configured to produce a shadow on the radiograph that indicates a center of the radiographic marker.

18. A radiograph, comprising:
a first shadow having a first shape, the first shadow having been placed on the radiograph in response to positioning a first portion of radiolucent material on a first surface of an imaging subject, the first portion of radiolucent material including a first radiopaque pattern that generates the first shadow when radiant energy is passed through the first portion of radiolucent material; and
a second shadow having a second shape that is visually distinguishable from the first shape, the second shadow having been placed on the radiograph in response to positioning a second portion of radiolucent material on a second opposite surface of the imaging subject, the second portion of radiolucent material including a second radiopaque pattern that is visually distinguishable from the first radiopaque pattern and that generates the second shadow when radiant energy is passed through the second portion of radiolucent material,
wherein the first shadow and the second shadow are usable for interpreting one or more characteristics of the subject as shown on the radiograph based on identification of the first shadow as corresponding to the first radiopaque pattern, based on identification of the second shadow as corresponding to the second radiopaque pattern, and based on a relative alignment of the first shadow and the second shadow.

19. The radiograph of claim 18, wherein the first shadow comprises a crosshair pattern, and wherein the second shadow comprises a target mark pattern.

20. The radiograph of claim 18, wherein the first shadow comprises a line pattern, and wherein the second shadow comprises a dot pattern.
